(19) United States
(12) Patent Application Publication
(10) Pub. No.: US 2008/0045912 A1
(43) Pub. Date: Feb. 21, 2008

(54) Sealing overflow safety trap

(76) Inventors: Steven J. Orwig, Bowie, MD (US); Kevin S. Bailey, Shady Side, MD (US); Ngee J. Lee, (US)

Correspondence Address: ROGER M. RATHBUN, 13 MARGARITA COURT, HILTON HEAD ISLAND, SC, 29926, US

(21) Appl. No.: 11/506,732

(22) Filed: Aug. 18, 2006

(51) Int. Cl. A61M 1/00

(52) U.S. Cl. 604/319

(57) ABSTRACT

A safety overflow trap for a medical suction system to withdraw liquids from a patient. The trap protects a vacuum regulator and other downstream components from contacting the liquid withdrawn from the patient. The trap has an inlet and an outlet and a reservoir intermediate thereto. A valve is provided between the reservoir and the outlet comprising a fixed member and a movable member. The movable member floats on the liquid accumulated in the reservoir and, as that liquid rises, the movable member rises to seal against the fixed member to occlude the source of vacuum. The fixed and movable members become latched together when the movable member enters the closed position to prevent the leakage of the liquid if the trap is inadvertently tipped over. A system is also present to provide a visual alert to the attending personnel when the movable member has moved to its closed position.

### FIELD OF THE INVENTION

The present invention relates to an overflow safety trap for medical suction systems and, more particularly, to a safety overflow trap that retains the fluids within a reservoir of the safety trap to avoid spillage from the overflow safety trap.

### BACKGROUND OF THE INVENTION

It is a common practice in the care of patients, particularly after surgery, to provide a vacuum system that carries out the withdrawal of liquids from the patient. To that end, most hospitals normally have a pipeline supply or source of vacuum that is piped to certain hospital rooms where the suction or withdrawal of liquids is being carried out. Alternatively, there may be an on-site source of vacuum actually within the hospital room itself that provides the necessary vacuum for withdrawal of the liquids.

In either event, there is normally also a vacuum regulator that is present within the treatment room so that the level of vacuum can be regulated by the caregiver to a particular level of vacuum that is desired, and of course, safe, to effectively apply to the particular cavity of a patient for the withdrawal of liquid therefrom.

One of the difficulties, however, in the use, for example, of a central hospital supply of the vacuum is that it is necessary to protect the vacuum regulator, as well as any further pipes, downstream equipment etc. from contamination by the biological liquids that are being removed from the patient. While the various components upstream of the vacuum regulator can be of a type that are readily cleaned or, alternatively, disposable, the downstream components and equipment including the vacuum regulator itself are of a nature that cleaning is very difficult or even not possible should the contaminated liquids actually enter the hospital piping of the central vacuum system.

Accordingly, such vacuum systems normally have some type of safety trap that is responsive to the presence of a liquid within a reservoir and, as the quantity of the fluid in that reservoir increases, there is a point where the amount of liquid reaches a maximum and a valve in the safety trap then occludes the vacuum line in some manner to prevent the liquid that has been collected in the reservoir from traveling further downstream of that safety device. In general, one current type of safety trap includes a fixed member and a movable member such that the movable member floats on the liquid accumulating in the reservoir and, as that liquid volume rises, the float rises on the liquid to the point were it interacts with the fixed member to shut off the supply of vacuum.

One problem with the present safety traps, however, is that the occluding of the vacuum line is not permanent, that is, if the safety trap is mishandled or simply tipped over, the movable member moves away from the fixed member and the valve thereupon opens and the liquid can pass through the then open valve and escape through the inlet and contaminate the surrounding area. Since the nature of the liquid that is accumulated in the reservoir is of a bio-hazardous nature, the leakage of that liquid is extremely undesirable.

Accordingly, it would be advantageous to have an overflow safety trap that contains the liquid within the reservoir after the valve has been closed despite a change in orientation of the safety trap, that is, once the movable member has sealed against the fixed member by means of the accumulation of an amount of liquid, the movable member interacts with the fixed member to retain the valve in a closed position such that, upon tipping over, that valve cannot again open to allow any of the liquid to escape through the inlet.

It would be further advantageous to have an overflow safety trap having a system to alert the attending personnel that the valve has moved to the closed position so that the attending personnel could promptly service the safety trap and return it to service to continue removing liquids from the patient.

### SUMMARY OF THE INVENTION

Accordingly, the present invention relates to a medical suction overflow safety trap that can be used in the vacuum line withdrawing liquids from a patient and provide protection for any components that are downstream of the device including the vacuum regulator. As used herein, the term upstream will refer to the direction of flow of the liquid in the vacuum system, thus, the patient is the ultimate upstream location and the source of vacuum is the ultimate downstream location.

With the protective device of the present invention, there is a manifold having an inlet and an outlet and which normally provides a fluid path for the vacuum that draws the liquids from the patient toward the vacuum regulator and accumulates the liquid in a reservoir that is located intermediate the inlet and the outlet. Thus, the present overflow safety trap is located upstream of the vacuum regulator, preferable adjacent that vacuum regulator and prevents the liquid from the patient from reaching the vacuum regulator. The outlet of the present protective device is thus adapted to be connected to the vacuum regulator and the inlet connected to the patient circuit including medical tubing that eventually leads to the patient via a collection bottle and the like.

A valve is located within the safety trap and which has a fixed member that is therefore affixed to the manifold and a movable member that basically floats on the surface of the liquid and, as the liquid rises through the accumulation of liquid within the reservoir, the movable member also rises to nest within an opening in the fixed member to shut of the communication between the inlet and the outlet, thereby shutting off the vacuum source and preventing the further withdrawal of liquid from the patient.

Once the movable member has moved to that closed position, there is an interaction or interfitting of the movable member and the fixed member to retain the movable member in the closed position. In an exemplary embodiment, the retention is accomplished through the presence of a generally J-shaped inwardly directed peripheral edge formed on the fixed member and which fits around a downward ledge shaped outer peripheral edge of the movable member.

Accordingly, once the movable member has been moved upwardly by the upwardly rising surface of the liquid and by the vacuum itself reaches its closed position, the inter-latching of the movable member to the fixed member retains the movable member in that closed position. As such, when the safety trap is later removed from the medical vacuum drainage system, it can be inadvertently tipped over or inverted during handling, and the bio-hazardous liquid will not spill out of the outlet to contaminate the surrounding area.

As a further feature, there is a system that alerts the attending personnel by a visual means where the movable member has moved to its closed position so that the personnel can promptly service the safety trap and return it to its function of removing liquids for that patient.

These and other features and advantages of the present invention will become more readily apparent during the following detailed description taken in conjunction with the drawings herein.

### BRIEF DESCRIPTION OF THE DRAWINGS

FIG. 1 is a schematic view of a sealing overflow safety trap constructed in accordance with the present invention with its valve in the open position;

FIG. 2 is a schematic view of a sealing overflow safety trap of FIG. 1 with its valve in the closed position;

FIG. 3 is a schematic view of the sealing overflow safety trap in the closed position of FIG. 2 resting on its side;

FIG. 4 is a cross-sectional view of a sealing overflow trap of the present invention;

FIG. 5A is a schematic view of an alternative embodiment of the sealing overflow safety trap of the present invention with its valve in the open position; and

FIG. 5B is a schematic view of the alternative embodiment of FIG. 5A with its valve in the closed position.

### DETAILED DESCRIPTION OF THE INVENTION

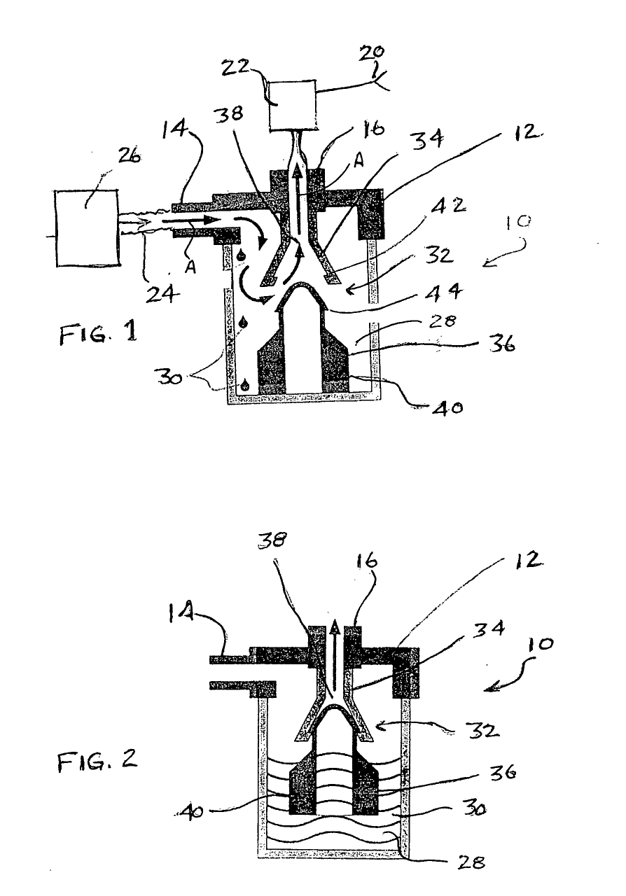

Referring now to FIG. 1, there is shown a schematic view of a sealing overflow safety trap 10 constructed in accordance with the present invention. As can be seen, the safety trap 10 comprises a manifold 12 having an inlet 14 and an outlet 16. The outlet 16 is adapted to be connected to a vacuum source 20 such as a pipeline central supply of vacuum in a hospital and the level of vacuum is controlled or set by means of a vacuum regulator 22.

The inlet 14 is, in turn, adapted to be connected to a tubing 24 that fluidly communicates with the area of the patient 26 that is being drained. As therefore can be seen, the liquid flows in the direction of the arrows A from the patient 26 toward the vacuum source 20.

There is a reservoir 28 located in a position between or intermediate the inlet 14 and the outlet 16. Thus the liquid 30 being withdrawn from the patient 26 enters the reservoir 28 and collects in the bottom thereof. As the liquid 30 collects and accumulates in the reservoir 28, the level of the liquid naturally rises.

The safety trap 10 also includes a valve 32 that is located intermediate the reservoir 28 and the outlet 16 and the valve 32 comprises a fixed member 34 and a movable member 36. The fixed member 34 is fixed with respect to the manifold 12 and the movable member 36 moves between an open position and a closed position where the flow of fluid between the reservoir 28 and the outlet 16 is closed, that is, the vacuum source 20 is basically occluded and no vacuum reaches the patient to continue with the withdrawal of liquids from the patient. As shown in FIG. 1, the movable member 36 is in its open position.

The fixed member 34 is basically an inverted cone-shaped configuration with a passageway 38 located at the top of the inverted cone shape that provides fluid communication between the reservoir 28 and the outlet 16, again as shown by the arrows A.

The movable member 36 has a bullet shaped upper surface that interfits into the inverted cone shape inner surface of the fixed member 34 and the movable member 36 has a number of fins 40 to stabilize it within the reservoir 28 as it moves therein. The movable member 36 is comprised of a light molded plastic material such that it floats on the surface of the liquid 30 as the liquid surface rises due to the accumulation of liquid within the reservoir 28.

Turning now to FIG. 2, taken along with FIG. 1, there is shown a schematic view of the safety trap 10 as described with respect to FIG. 1 but with the movable member 36 in its closed position. Thus, the level of the liquid 30 has risen due to the continued accumulation of the liquid withdrawn from the patient 26 such that the floating movable member 36 has been elevated upwardly to enter into and seal against the fixed member 34, thereby occluding the passageway 38 and effectively cutting off the supply of vacuum.

At this point, therefore, the reservoir 28 has accumulated the maximum amount of liquid feasible and the safety trap 10 has shut off the supply of vacuum from the vacuum source 20 to prevent any of the bio-hazardous liquid 30 from entering the passageway 38 and possibly reaching the vacuum regulator 22.

There can also be seen, an inner peripheral formation 42 generally in the shape of a lip that formed is on the internal surface of the fixed member 34. There is also an outer peripheral formation 44 formed on the external surface of the movable member 36 and, as can be seen in FIG. 2, the inner peripheral formation 42 engages and captures the outer peripheral formation 44 such that the movable member 36 is firmly retained in its closed position joined to the fixed member 34 and the passageway 38 remains sealed.

Turning now to FIG. 3, there is shown a schematic view of the safety trap 10 and it can be seen that the safety trap 10 is laying on its side as if it has been tipped over. The inadvertent tipping over of the safety trap 10 is common and can occur when the safety trap 10 has been removed from the medical vacuum system for maintenance such as cleaning or the like. As such, in FIG. 3, it can be seen that but for the movable member 36 being retained in its closed position sealing against the fixed member 34, there would have leakage of the bio-hazardous liquid out of the safety trap 10 through the outlet 16 to the surrounding environment, thereby contaminating that environment.

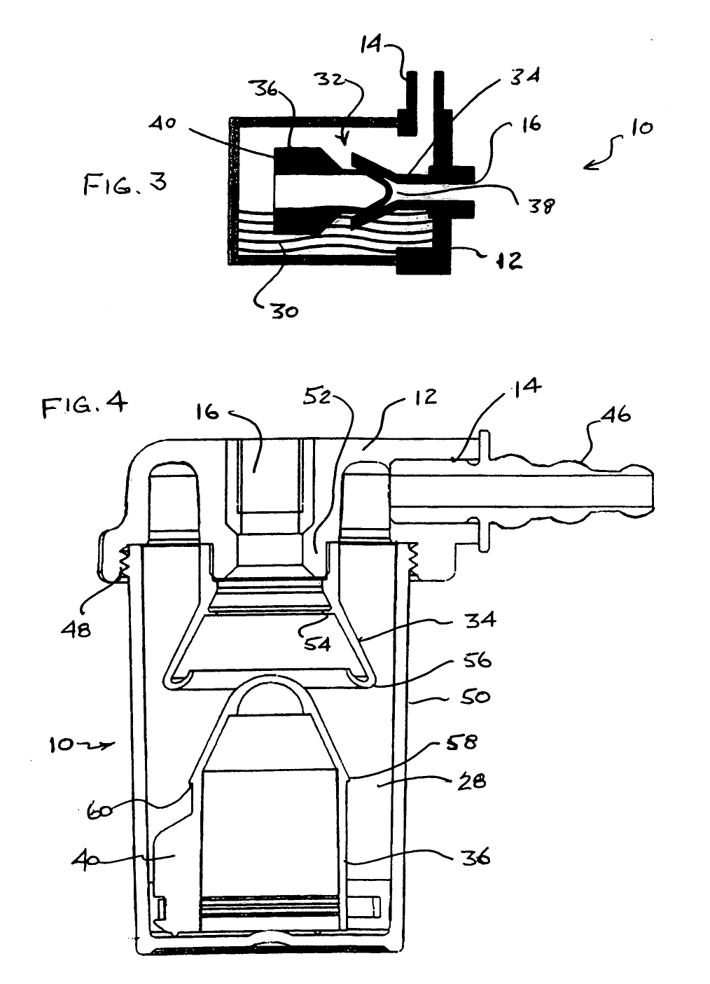

Turning now to FIG. 4, there is shown a cross sectional view of a safety trap 10 of the present invention and like numbers will be used to explain FIG. 4 as were used in describing FIGS. 1-3 where the same components are concerned. As such there are four main components; the manifold 12, the reservoir 28, the fixed member 34 of the valve 32 and the movable member 36 of the valve 32.

The manifold 12 is a plastic molded component and the inlet 14 has a fitting 46 for connecting to a flexible tubing that communicates with the area of the patient to be drained. The underside of the manifold 12 has threads 48 that receive a similarly threaded molded plastic container 50 to form the reservoir 28 such that the plastic container 50 can be easily removed and replaced.

The fixed member 34 is a molded plastic component that is affixed to a boss 52 formed in the underside of the manifold 12. There may also be an internal circular rib 54 formed on the fixed member 34 to aid in sealing against the movable member 36 when the movable member 36 is in its closed position. At the lower end of the fixed member 34, there is an inwardly directed edge 56, having the configuration of a J cross section and which surrounds the entire lower periphery of the fixed member 34.

Likewise, there is an outer peripheral edge 58 formed on the movable member 36 that forms a ledge 60 and, as can be seen, the inwardly directed edge 56 of the fixed member 34 can encompass and surround the outer peripheral edge 58 of the movable member 36 to retain that movable member 36 in its closed, sealed position with respect to the fixed member 34.

Accordingly, in the operation of the overflow safety trap 10, as the liquid accumulates in the reservoir 28, the movable member 36 rises along with the rising of that liquid. When sufficient liquid has accumulated, the movable member enters into the fixed member 34 and seals therein with the internal circular rib 54 aiding in that sealing so that the outlet 14 is closed and the vacuum source thereby shut off. Thus, the movable member 36 is then in its closed position and the inwardly directed edge 56 of the fixed member 34 latches with the peripheral edge 58 of the movable member 36 to retain the movable member 36 securely fastened to the fixed member 34.

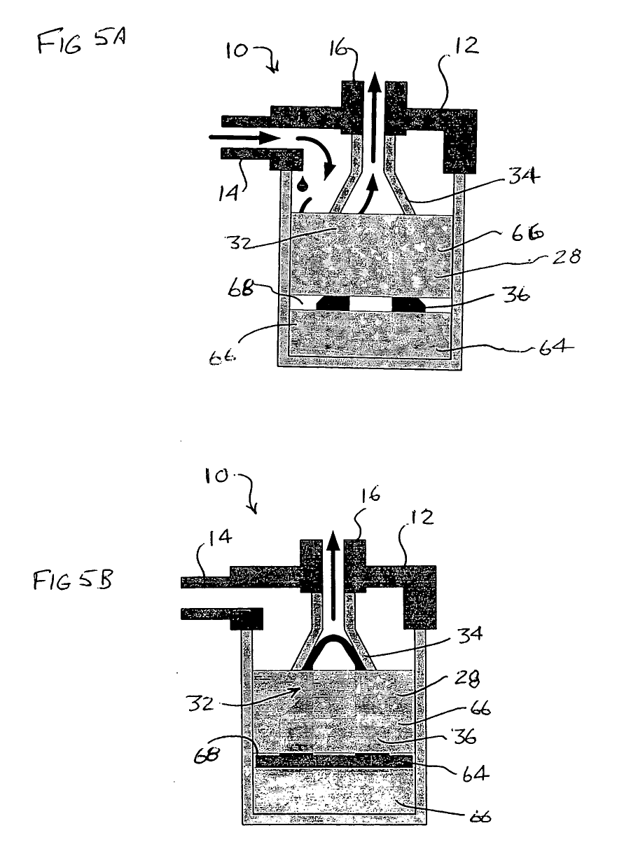

Turning now to FIGS. 5A and SB, there is shown schematic views of an alternative embodiment of the present sealing overflow safety trap 10 and again, like numbers will be used corresponding to the same components explained with reference to FIGS. 1 and 2. Again, the safety trap 10 comprises a manifold 12 having an inlet 14 and an outlet 16 adapted to be connected to a controllable source of vacuum.

In FIG. 5A, the valve 32 is open since the movable member 36 is separated away from the fixed member 34. In this embodiment there is an indicating area 64 that is preferably formed as part of the movable member 36 or is provided on the movable member 36 and which is brightly colored, such as in a red color.

As can also be seen in the FIGS. 5A and 5B, a substantial portion of the plastic container 50 is a non-transparent area 66, such as a semi-opaque area such that there is some visibility through the material to view the internal reservoir 28 but that visibility is obscured or impaired. There is a transparent window 68 formed in that non-transparent area 66 at a predetermined location. As such, in FIG. 5A, with the valve 32 in its open position, the indicating area 64 is obscured from the vision of attending personnel since it is located within the non-transparent area 66 such that the brightly colored indicating member is not readily visible.

When, however, the valve 32 is in its closed position where the movable member 36 is engaged with the fixed member 34, that movable member 36 has moved vertically upwardly such that the brightly colored indicating area 64 is aligned with the transparent window 68 and thereby provides a very visible alert to the attending personnel that the reservoir 28 has accumulated sufficient liquid to cause the valve 32 to close so that the attending personnel can take the appropriate measures to restore the sucking action of the vacuum system to continue the drainage of the patient.

Those skilled in the art will readily recognize numerous adaptations and modifications which can be made to the safety overflow trap of the present invention which will result in an improved medical vacuum system, yet all of which will fall within the scope and spirit of the present invention as defined in the following claims. Accordingly, the invention is to be limited only by the following claims and their equivalents.

What is claimed is:

1. A safety overflow trap for use with a medical suction system, the safety overflow trap comprising a manifold having a outlet adapted to be connecting to a source of suction and an inlet adapted to be connected to a source of liquid, a reservoir fluidly located intermediate the inlet and the outlet for collecting liquid received through the inlet, and a valve located intermediate the reservoir and the outlet, the valve having a fixed member and a movable member that moves between an open position displaced away from the fixed member and a closed position wherein the movable member seats against the fixed member to prevent fluid from reaching the outlet, the improvement wherein the movable member is retained in the closed position by an interaction between the movable member and the fixed member.

2. The safety overflow trap of claim 1 wherein the movable member floats on the liquid received within the reservoir to move toward its closed position.

3. The safety overflow trap of claim 1 wherein the fixed member is an inverted cup shaped cone having an inner peripheral edge that interfits with the movable member to retain the movable member in the closed position.

4. The safety overflow trap wherein the movable member is a float having an outer peripheral edge that interfits with the inner peripheral edge of the fixed member.

5. The safety overflow trap of claim 3 wherein the inner peripheral edge of the fixed member is a J-shaped cross sectional configuration.

6. The safety overflow trap of claim 5 wherein outer peripheral edge of the movable member forms a downward ledge and the J-shaped inner peripheral edge of the fixed member engages the downward ledge of the movable member to retain the movable member in the closed position.

7. A medical suction system for withdrawing liquids from a patient, the system comprising:
a vacuum regulator adapted to be connected to a source of vacuum,
a safety overflow trap located intermediate a patient and the vacuum regulator, said safety overflow trap comprising a manifold having an outlet fluidly connected to the vacuum regulator and an inlet adapted to be connected to a patient, and a reservoir intermediate the inlet and the outlet for collecting liquid,
the safety overflow trap having a valve comprising a fixed member and a movable member that moves between an open position displaced away from the fixed member and a closed position where the movable member seats against the fixed member to prevent liquid from reaching the outlet and wherein the movable member and the fixed member engage each other to retain the movable member in the closed position.

8. The system of claim 7 wherein the movable member is moved toward its closed position by means of the liquid that is collected in the reservoir.

9. The system of claim 8 wherein the movable member floats on the liquid collected in the reservoir.

10. The system of claim 7 wherein the fixed member is an inverted cup shaped cone having an inner peripheral edge that interfits with the movable member to retain the movable member in the closed position.

11. The system of claim 10 wherein the movable member is a float having an outer peripheral edge that interfits with the inner peripheral edge of the fixed member.

12. The system of claim 10 wherein the inner peripheral edge of the fixed member is a J-shaped cross sectional configuration.

13. The system of claim 12 wherein the outer peripheral edge of the movable member forms a downward ledge and the J-shaped inner peripheral edge of the fixed member engages the downward ledge of the movable member to retain the movable member in the closed position.

14. A method of providing protection against liquid passing through a medical suction system, said method comprising the steps of:
providing a vacuum regulator adapted to be connected a source of vacuum,
providing a safety overflow trap upstream of the vacuum regulator and in fluid communication therewith, the safety overflow trap having an inlet for receiving liquid from a patient and an outlet wherein gas normally flows through said outlet to the vacuum regulator,
providing a reservoir intermediate the inlet and the outlet for collecting the liquids from a patient,
providing a valve intermediate the reservoir and the outlet, the valve having a fixed member and a movable member that seats against the fixed member to provide a closed position occluding the flow to the vacuum regulator when a quantity of liquid has accumulated in the reservoir and,
retaining the valve in the closed position by an interfitting of the movable member with the fixed member.

15. The method of claim 14 wherein the step of retaining the valve in the closed position comprises providing a peripheral formation on the fixed member that attaches to the movable member when the movable member moves to the closed position.

16. The method of claim 15 wherein the step of retaining the valve in the closed position comprises providing a peripheral formation on the movable member that interfits with the peripheral formation on the fixed member when the movable member moves to the closed position.

17. The method of claim 14 wherein the step of providing a valve comprises providing a valve where the movable member is moved to its closed position by the accumulation of liquid in the reservoir.

18. The method of claim 17 wherein the step of providing a valve comprises providing a valve where the movable member floats on the liquid accumulating in the reservoir.

19. A safety overflow trap for use with a medical suction system, the safety overflow trap comprising a manifold having a outlet adapted to be connecting to a source of suction and an inlet adapted to be connected to a source of liquid, a reservoir fluidly located intermediate the inlet and the outlet for collecting liquid received through the inlet, and a valve located intermediate the reservoir and the outlet, the valve having a fixed member and a movable member that moves between an open position displaced away from the fixed member and a closed position wherein the movable member seats against the fixed member to prevent fluid from reaching the outlet, the reservoir being comprised of a non-transparent material having a transparent window formed therein, said movable member having a brightly colored indicating area, said indicating area being aligned with the non-transparent material when the movable member is in the open position and wherein the indicating area is aligned with the transparent window when the movable member is in the closed position.

20. The safety overflow trap of claim 19 where the non-transparent material is a semi-opaque material.
